(19) United States
(12) Patent Application Publication
(10) Pub. No.: US 2025/0295878 A1
(43) Pub. Date: Sep. 25, 2025

(54) MASK AUTO-IDENTIFICATION VIA BREATHING SOUND CLASSIFICATION

(71) Applicant: KONINKLIJKE PHILIPS N.V., EINDHOVEN (NL)

(72) Inventors: CORNELIS PETRUS HENDRIKS, EINDHOVEN (NL); OKKE OUWELTJES, VELDHOVEN (NL); JOACHIM JOHANNES KAHLERT, AACHEN (DE); JOYCE VAN ZANTEN, WAALRE (NL); DAAN ANTON VAN DEN ENDE, BREDA (NL)

(21) Appl. No.: 19/086,619

(22) Filed: Mar. 21, 2025

Related U.S. Application Data
(60) Provisional application No. 63/569,464, filed on Mar. 25, 2024.

(51) Int. Cl. A61M 16/00; A61M 16/06

(52) U.S. Cl. CPC A61M 16/026; A61M 16/06; A61M 2205/3375; A61M 2205/6018; A61M 2230/42

(57) ABSTRACT

A system and associated method for automatically identifying a mask used in a pressure support system for delivering a flow of breathing gas to the airway of a patient. The system includes a controller implementing a trained machine learning model. The controller is structured and configured to receive a sound signal, the sound signal being indicative of breathing sounds (e.g., exhalation and/or inhalation sounds) captured from the patient during use of the mask in the pressure support system, generate acoustic spectrum data indicative of an acoustic spectrum of the exhalation sounds based on the sound signal, provide the acoustic spectrum data to the trained machine learning model, and determine a brand, type and/or size of the mask in the trained machine learning model based on the provided acoustic spectrum data.

### CROSS-REFERENCE TO RELATED APPLICATIONS

This patent application claims the priority benefit under 35 U.S.C. § 119(e) of U.S. Provisional Application No. 63/569,464, filed on Mar. 25, 2024, the contents of which are herein incorporated by reference.

### BACKGROUND OF THE INVENTION

### 1. Field of the Invention

The disclosed concept relates generally to pressure support systems, and, more particularly, to a pressure support system in which breathing sounds, such as exhalation and/or inhalation sounds, are utilized to automatically identify the mask being used in the system.

### 2. Description of the Related Art

Today, the first line therapy for patients diagnosed with obstructive sleep apnea syndrome (OSAS) after a sleep test is pressure assisted ventilation support, also known as PAP support or therapy, most often by continuous positive airway pressure (CPAP) therapy. Such pressure assisted ventilation support involves the placement of a respiratory patient interface device, including a mask component, on the face of a patient. The mask component may be, for example and without limitation, a nasal mask that covers the patient's nose, a nasal cushion having nasal prongs that are received within the patient's nares, a pillow-style nasal cushion that engages the patient's nares without being inserted therein, a nasal/oral mask that covers the patient's nose and mouth, or a full face mask that covers the patient's face. The respiratory patient interface device interfaces a pressure/flow generating device (also known as a PAP device) with the airway of the patient so that a flow of breathing gas can be delivered from the pressure/flow generating device to the airway of the patient.

In moderate and severe OSAS patients with an apnea-hypopnea index (AHI) that is greater than 15, the therapy is reimbursed. In mild OSAS patients with daytime symptoms, or with chronic and persistent cardiac comorbidities, the PAP therapy is reimbursed for an AHI that is greater than 5. Reimbursement covers the PAP device as well as periodic resupply of consumable items, such as tubing, headgear, masks, and cushions. Depending on the geography, different time periods for replacement of these consumable items are in effect, mostly ranging from 1 month to 6 months.

The proper setup of the PAP device including, for instance, pressure settings and fitting of the mask, is done by a qualified sleep clinician, most often in an overnight setting at a sleep lab, although home titration is a possible alternative for certain patients. Once the correct machine settings and appropriate consumable items have been established, the equipment is supplied by a durable medical equipment (DME) supplier and the patient commences therapy.

When the mask is due for replacement, the DME may supply a new mask to the patient. Often, DMEs send their patients not just one mask but a pack that contains all available mask sizes (a so-called “fit pack”), since it is easier for DMEs to send such a pack which contains all mask sizes when they do not know exactly the mask size that the patient is using. Patients can use any of the masks in this pack after receiving it and may switch mask sizes without the clinician or DME knowing which mask they are using. Also, patients or DMEs may replace the mask with a different type of mask than was originally prescribed. In some cases. patients might even switch mask brands without the clinician or DME knowing.

The identification of the actual mask type, size and/or brand that is used during therapy at any time is a need that is expressed by clinicians and DMEs alike. This would allow clinicians and/or DMEs to improve the understanding of collected CPAP data and diagnose problems more accurately (especially in a remote/telehealth setting), to manage resupply, reimbursement, and product lifetime, and to remotely control device settings depending on the type of mask that is used.

### SUMMARY OF THE INVENTION

In one embodiment, a system for automatically identifying a mask used in a pressure support system for delivering a flow of breathing gas to the airway of a patient is provided. The system includes a controller, the controller implementing a trained machine learning model. The controller is structured and configured to receive a sound signal, the sound signal being indicative of breathing sounds (e.g., exhalation and/or inhalation sounds) captured from the patient during use of the mask in the pressure support system, generate acoustic spectrum data indicative of an acoustic spectrum of the breathing sounds based on the sound signal, provide the acoustic spectrum data to the trained machine learning model, and determine a brand, type and/or size of the mask in the trained machine learning model based on the provided acoustic spectrum data.

In another embodiment, a method for automatically identifying a mask used in a pressure support system for delivering a flow of breathing gas to the airway of a patient is provided. The method includes receiving a sound signal, the sound signal being indicative of breathing sounds (e.g., exhalation and/or inhalation sounds) captured from the patient during use of the mask in the pressure support system, generating acoustic spectrum data indicative of an acoustic spectrum of the breathing sounds based on the sound signal, providing the acoustic spectrum data to a trained machine learning model, and determining a brand, type and/or size of the mask in the trained machine learning model based on the provided acoustic spectrum data.

### BRIEF DESCRIPTION OF THE DRAWINGS

A full understanding of the invention can be gained from the following description of the preferred embodiments when read in conjunction with the accompanying drawings in which:

FIG. 1 is a schematic diagram of a pressure support system in which breathing sounds are used to automatically identify the mask used in the system according to an embodiment of the disclosed concept;

FIG. 2 is a block diagram of an electronics module forming part of the pressure generating device of the system shown in FIG. 1;

FIG. 3 is a schematic diagram of a pressure support system in which breathing sounds are used to automatically identify the mask used in the system according to an alternative embodiment of the disclosed concept; and

FIG. 4 is a block diagram of an electronics module forming part of the pressure generating device of the system shown in FIG. 3.

### DETAILED DESCRIPTION OF THE INVENTION

As used herein, the singular form of “a”, “an”, and “the” include plural references unless the context clearly dictates otherwise.

As used herein, the statement that two or more parts or components are “coupled” shall mean that the parts are joined or operate together either directly or indirectly, i.e., through one or more intermediate parts or components, so long as a link occurs.

As used herein, “directly coupled” means that two elements are directly in contact with each other.

As used herein, the term “number” shall mean one or an integer greater than one (i.e., a plurality).

As used herein, the term “controller” shall mean a programmable analog and/or digital device (including an associated memory part or portion) that can store, retrieve, execute and process data (e.g., software routines and/or information used by such routines), including, without limitation, a field programmable gate array (FPGA), a complex programmable logic device (CPLD), a programmable system on a chip (PSOC), an application specific integrated circuit (ASIC), a microprocessor, a microcontroller, a programmable logic controller, or any other suitable processing device or apparatus. The memory portion can be any one or more of a variety of types of internal and/or external storage media such as, without limitation, RAM, ROM, EPROM(s), EEPROM(s), FLASH, and the like that provide a storage register, i.e., a non-transitory machine readable medium, for data and program code storage such as in the fashion of an internal storage area of a computer, and can be volatile memory or nonvolatile memory.

As used herein, the term “spectrogram” shall mean a visual representation of the spectrum of frequencies of an audio signal as it varies with time.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

The disclosed concept, as described in detail herein, provides a pressure support system and associated method wherein the mask of the pressure support system is able to be automatically identified using breathing sounds, such as exhalation and/or inhalation sounds, obtained from the user of the pressure support system during use. As described in detail herein in connection with various exemplary embodiments, the system of the disclosed concept includes a PAP device, a patient interface device including a mask, a microphone, and a controller. The controller is provided with and is configured to execute an algorithm for classifying the mask type, size and/or brand based on the acoustic spectrum of the gas being exhaled and/or inhaled by the user through the mask during use of the system. The microphone that is part of the system and method may come from any of a number of sources, as suitable microphones are available in many parts of the current ecosystem (e.g., in smartphones, wearables, wake-up lights, earplugs, smart home systems, etc.).

According to the disclosed concept, the recorded breathing sound signal (e.g., in the form of a suitable audio file) is converted into a spectrogram. The spectrogram may, for example and without limitation, be calculated with a Short-Term Fourier Transform (STFT), or using Mel-frequency cepstral coefficients (MFCC). In the exemplary embodiment, the algorithm that classifies the mask type, size and/or brand based on the acoustic spectrum employs a trained machine learning model, which is in one embodiment a trained convolutional neural network (CNN). More specifically, the trained machine learning model is configured to receive the spectrogram and its features as an input and to classify the mask type, size, and/or brand based on that input. In an alternative embodiment, the algorithm that classifies the mask type and size may use signal correlation techniques wherein measured acoustic signals are correlated with stored acoustic signals.

The data acquisition and processing of the breathing sound signal according to the disclosed concept may be performed on the PAP system, on a local (e.g. mobile) computing device such as smartphone or tablet computer, or on a remote computing device such as a cloud server or edge device (or some combination thereof). The data acquisition and processing may be activated based on user input, for example with an “identify mask” push button, or may be activated automatically each time the PAP device is powered on. The output of the algorithm may be uploaded and displayed in a computerized sleep and respiratory care management system, such as the Care Orchestrator system provided by Koninklijke Philips N.V., in the form a warning (“mask changed”) or as a piece of information (e.g., mask type, size, brand) used by the sleep and respiratory care management system.

The advantages of the solution of the disclosed concept are many, and include, without limitation, ease of use and implementation (microphones are everywhere), lack of a need for additional system hardware, components or patient interface modifications, and the brand agnostic nature of the solution.

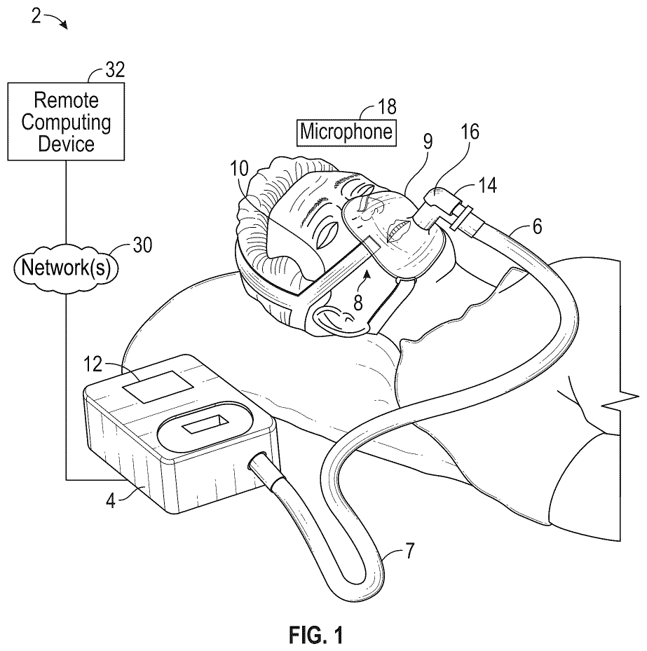

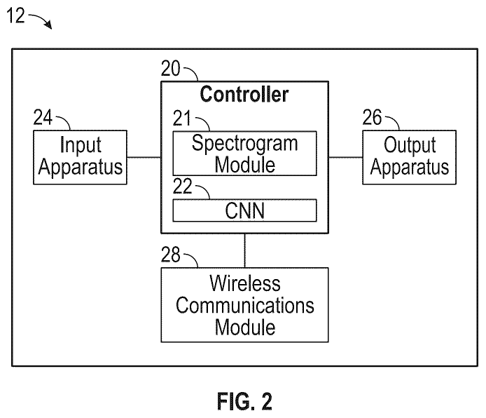

FIG. 1 is a schematic diagram of a pressure support system 2 in which exhalation sounds are used to automatically identify the mask used in the system according to a non-limiting exemplary embodiment of the disclosed concept. While the exemplary embodiments described herein use exhalation sounds for illustration purposes, it will be understood that this is meant to be exemplary only and that inhalation sounds may be used in lieu of or in addition to exhalation sounds within the scope of the disclosed concept. Pressure support system 2 is adapted to provide a regimen of respiratory therapy to a patient. Pressure support system 2 includes a pressure generating device 4 (also known as a PAP device) and a patient circuit 6 including a delivery conduit 7 and a patient interface device 8. In the illustrated embodiment, patient interface device 8 includes a mask 9 (having a flexible cushion and, optionally, a rigid faceplate) and a headgear 10 for securing mask 9 to the head of the patient. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Koninklijke Philips N.V.), and auto-titration pressure support devices. As seen in FIG. 1, pressure generating device 4 includes an electronics module 12, which is described in greater detail herein in connection with FIG. 2.

Delivery conduit 7 is structured to communicate the flow of breathing gas from pressure generating device 4 to patient interface device 8. Typically, delivery conduit 7 includes one or more individual conduits or tubes, a first end of which couples with pressure generating device 4 and a second end of which couples with patient interface device 8. In the illustrated embodiment, the second end is coupled with patient interface device 8 through a fluid coupling device 14 (e.g., an elbow conduit) of patient interface device 8.

In the exemplary embodiment illustrated in FIG. 1, mask 9 is a nasal/oral mask structured to be placed on the face of a patient. Any type of mask 9, however, which facilitates the delivery of the flow of breathing gas to, and the removal of a flow of exhalation gas from, the airway of such a patient may be used while remaining within the scope of the disclosed concept. An opening in the mask 9, to which fluid coupling device 14 is coupled, allows the flow of breathing gas from pressure generating device 4 to be communicated to an interior space defined by mask 9, and then to the airway of the patient. The opening in mask 9 also allows the flow of exhalation gas from the airway of the patient to be communicated to an exhaust port assembly 16 provided in fluid coupling device 14.

As seen in FIG. 1, pressure support system 2 further includes a microphone 18 for collecting exhalation sounds from the patient as exhaled gases are vented through exhalation port 16 while the patient is using pressure support system 2.

Microphone 18 may come from any part of the ecosystem surrounding pressure support system 2. For example, and without limitation, microphone 18 may form part of a smartphone or tablet computer of the patient or a caregiver. In addition, the sound signals captured by microphone 18 may be provided to electronics module 12 described below using a wired or wireless connection. For example, if microphone 18 comes from a smartphone or tablet computer having wireless communications capabilities, the captured sound signals may be wirelessly transmitted to electronics module 12 for processing thereby, in particular by a trained convolutional neural network (CNN) 22 described below.

FIG. 2 is a block diagram of electronics module 12 according to an exemplary embodiment of the disclosed concept. Electronics module 12 includes a controller 20, an input apparatus 24 (such as a keyboard), and an output apparatus 26 (such as a liquid crystal display). A user is able to provide input into controller 20 using input apparatus 24, and controller 20 provides output signals to output apparatus 26 to enable output apparatus 26 to display information to the user. Controller 20 implements a spectrogram module 21 that is configured for receiving the exhalation sounds that are emanated from exhalation port 16 and captured by microphone 18 and for generating a spectrogram based on the received exhalation sounds. As noted elsewhere herein, spectrogram module 21 may calculate spectrograms using, for example and without limitation, a Short-Term Fourier Transform (STFT), Mel-frequency cepstral coefficients (MFCC), or any other suitable method. Controller 20 also implements a trained convolutional neural network (CNN) 22 (or another type of suitable machine learning model) for automatically identifying the mask used in pressure support system 2 based on the spectrogram generated by spectrogram module 21. More specifically, the memory portion of controller 20 has stored therein a number of routines (comprising computer executable instructions) that are executable by the processor portion of controller 20, including routines for implementing spectrogram module 21 and trained CNN 22 as described herein. Electronics module 12 further includes a wireless communications module 28 for enabling electronics module 12 to wirelessly communicate with other devices, such as the device containing microphone 18 and/or remote computing device 32 described below.

The power spectrum of sound originating from exhalation ports 16 will depend on the particulars of the structure and materials of mask 9. CNN 22 in the exemplary embodiment (or another suitable machine learning model) can be trained to classify the type, size and/or brand of mask 9 using acoustic data collected in sleep labs or during home sleep tests (using patient populations with different masks and device settings). CNN 22 may also be improved with new incoming data (while patients are using system 2). Ground truth for the training may be provided by limiting the patient use data to data from nights that are verified for the correct mask (for instance by asking whether the patient is still using the original mask) and/or by using data from only the nights before a follow up lab visit or DME telephone follow up where the patient is asked by staff which mask they used. This limits the incoming data but is advantageous to preserve quality. Alternatively, the learning algorithm may randomly ask patients to submit their mask ID (in form of QR code or other common way) to verify the acoustic data with ground truth.

One challenge for the disclosed concept is the need to classify multiple brands and mask types and sizes. In one example, it may be necessary to classify 6 brands, 5 mask types, and 3 mask sizes, meaning a total of 90 classes or categories. An additional complication is the interaction between the patient and the mask and between the pressure and the mask. In general, the number of classes/categories a classifier can classify with good precision is determined by: (i) how distinct each category is, (ii) how many features can be derived from the content, and (iii) how many high-quality labeled examples are available. In principle, it is possible to build a CNN, such as CNN 22, with 90 outputs (labels) or more. For example, in known car recognition application using a CNN, 196 classes of cars have been able to be distinguished using a car data set consisting of 16,000 images (8000 for training and 8000 for testing).

A practical problem is how to get such an amount of training data for the disclosed concept. One option is a large study with thousands of patients. Another option is to gather data from clinical practice using sound recordings during therapy set-up when the mask type and size is known. Alternatively, data from later-on in the therapy can be used when the mask has been changed (consciously and known by the patient and therapist). An option to improve the model is to ask questions such as “did you change your mask?”, or “are you now using mask X?” when a change in spectrogram is detected.

One way to quickly generate a large amount of training data is to use in silico data which can be generated with acoustical simulations. For example, a computational fluid dynamics (CFD) analysis of the sound generated by different masks and sizes, optionally in different conditions (e.g., different breathing waveforms, device pressures, etc.). An additional challenge is potential disturbances, such as the sound from un-intentional mask leak or sound from the environment (e.g., from an air conditioner or fan). This can be mitigated with filtering, for example, by subtracting the background noise (spectrum) from the signal (spectrum).

As seen in FIG. 1, in the disclosed concept, pressure generating device 4 is coupled to one or more communications networks 30. A remote computing device 32, such as a server computer or an edge device, is also coupled to the network(s) 30. In the exemplary embodiment, remote computing device 32 is a computerized sleep and respiratory care management system, such as the Care Orchestrator system provided by Koninklijke Philips N.V. Thus, the mask identification that is performed by CNN 22 as described herein can be communicated to remote computing device 32 so that information relating thereto can be provided to a caregiver or a DME. As noted elsewhere herein, that information may be in the form a warning (“mask changed”) or as a piece of information (mask type, size, and/or brand) that is used by the sleep and respiratory care management system.

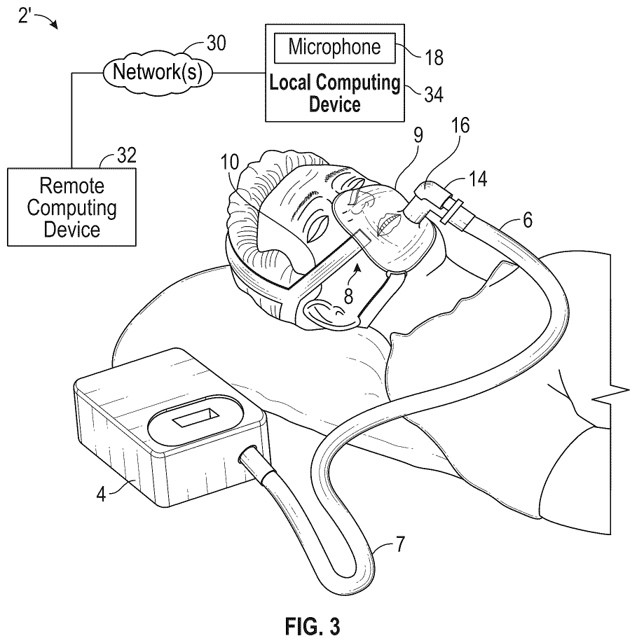

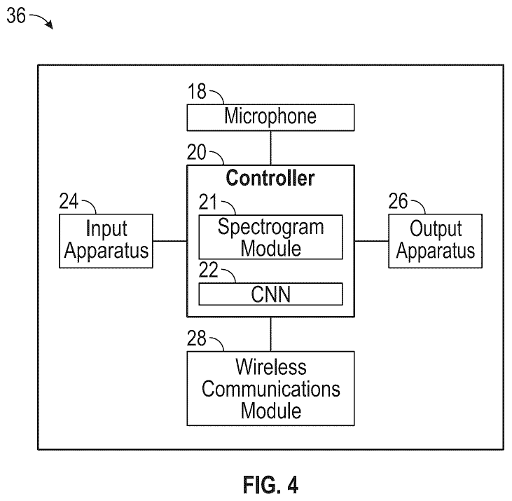

FIG. 3 is a schematic diagram of a pressure support system 2′ in which exhalation sounds are used to automatically identify the mask used in the system according to an alternative non-limiting exemplary embodiment of the disclosed concept. Again, while the exemplary embodiments described herein use exhalation sounds for illustration purposes, it will be understood that this is meant to be exemplary only and that inhalation sounds may be used in lieu of or in addition to exhalation sounds within the scope of the disclosed concept. Pressure support system 2′ is similar to pressure support system 2, and like components are labeled with like reference numerals. However, in pressure support system 2′, microphone 18 is provided as part of a local computing device 34, such as, without limitation, a smartphone or a tablet computer. In this embodiment, it is local computing device 34 rather than pressure generating device 4 that generates the spectrogram based on the exhalation sounds emanating from exhalation ports 16 and that identifies the type, size and/or brand of mask 9 based thereon. More specifically, as seen in FIG. 4, local computing device 34 includes an electronics module 36 that is similar to electronics module 12 and that includes controller 20 that implements spectrogram module 21 and CNN 22 as described herein. The mask identification that is performed by CNN 22 as described herein in this embodiment can be communicated to remote computing device 32 via network(s) 30 so that information relating thereto can be provided to a caregiver or a DME.

According to still a further alternative embodiment, the sound signals indicative of the exhalation sounds captured by microphone 18 may be provided to remote computing device 32. In this further alternative embodiment, it is remote computing device 32 that implements spectrogram module 21 and CNN 22 as described herein for identifying the type, size and/or brand of mask 9 based on patient exhalation sounds.

The sensitivity/specificity of the determination made according to the disclosure concept may, in further alternative exemplary embodiments, be improved by adding additional signals or information to the mask auto-identification processing, such as, without limitation, device flow and pressure data, mask fit data from, for example, a computerized mask selector tool (e.g., patient facial geometry, mask fit loose or snug). Also, the sound of the PAP device can be used to filter the sound signal before the acoustic spectrum data is determined by filtering out the pump frequency of the blower of the PAP device. In addition, the acoustic spectrum data may be measured as a function of the patient's breathing cycle and fed to the CNN.

In still further non-limiting exemplary embodiments, the acoustic spectrum of the specific patient-mask combination is characterized during mask fit. When the spectrum alters during usage, this indicates a change in mask type or size. This causes an alert and/or or a request to enter the new mask type and size. This embodiment does not require model training; a correlation algorithm can classify the spectrogram or signal (“significantly changed or not changed”). In addition, in this embodiment, the patient may be asked to speaking while wearing a mask (full face only) by, for example, reading aloud a specific sentence during mask fit. Such a feature will further improve the sensitivity and specificity of the algorithm.

While specific embodiments of the invention have been described in detail, it will be appreciated by those skilled in the art that various modifications and alternatives to those details could be developed in light of the overall teachings of the disclosure. Accordingly, the particular arrangements disclosed are meant to be illustrative only and not limiting as to the scope of disclosed concept which is to be given the full breadth of the claims appended and any and all equivalents thereof.

What is claimed is:

1. A system for automatically identifying a mask used in a pressure support system for delivering a flow of breathing gas to the airway of a patient, comprising:
a controller, the controller implementing a trained machine learning model, the controller being structured and configured to:
receive a sound signal, the sound signal being indicative of breathing sounds captured from the patient during use of the mask in the pressure support system;
generate acoustic spectrum data indicative of an acoustic spectrum of the breathing sounds based on the sound signal;
provide the acoustic spectrum data to the trained machine learning model; and
determine a brand, type and/or size of the mask in the trained machine learning model based on the provided acoustic spectrum data.

2. The system according to claim 1, wherein the acoustic spectrum data comprises a spectrogram.

3. The system according to claim 1, wherein the trained machine learning model comprises a trained convolutional neural network.

4. The system according to claim 1, wherein the controller is part of a computing device that is remote from the pressure support system.

5. The system according to claim 1, wherein the controller is configured to send a message to a computing device that is remote from the pressure support system, wherein the message is based on the determination of the brand, type and/or size of the mask.

6. A method for automatically identifying a mask used in a pressure support system for delivering a flow of breathing gas to the airway of a patient, comprising:
receiving a sound signal, the sound signal being indicative of breathing sounds captured from the patient during use of the mask in the pressure support system;
generating acoustic spectrum data indicative of an acoustic spectrum of the breathing sounds based on the sound signal;
providing the acoustic spectrum data to a trained machine learning model; and
determining a brand, type and/or size of the mask in the trained machine learning model based on the provided acoustic spectrum data.

7. The method according to claim 6, wherein the acoustic spectrum data comprises a spectrogram.

8. The method according to claim 6, wherein the trained machine learning model comprises a trained convolutional neural network.

9. The method according to claim 6, wherein the trained machine learning model is implemented on a controller of the pressure generating device of the pressure support system.

10. The method according to claim 6, wherein the trained machine learning model is implemented on a computing device that is remote from the pressure support system.

11. The method according to claim 6, further comprising sending a message to a computing device that is remote from the pressure support system, wherein the message is based on the determination of the brand, type and/or size of the mask.

12. The system according to claim 1, wherein the sound signal is formed by a process including filtering out a pump frequency of a pressure generating device of the pressure support system from a captured sound signal.

13. The method according to claim 6, wherein the sound signal is formed by a process including filtering out a pump frequency of a pressure generating device of the pressure support system from a captured sound signal.

14. The system according to claim 1, wherein pressure and/or flow data from use of the pressure support system by the patient is provided to the trained machine learning model, and wherein the brand, type and/or size of the mask is determined in the trained machine learning model based on the provided acoustic spectrum data and the pressure and/or flow data.

15. The method according to claim 6, wherein pressure and/or flow data from use of the pressure support system by the patient is provided to the trained machine learning model, and wherein the brand, type and/or size of the mask is determined in the trained machine learning model based on the provided acoustic spectrum data and the pressure and/or flow data.

16. The system according to claim 1, wherein the acoustic spectrum data indicative of the acoustic spectrum of the breathing sounds based on the sound signal is a function of a breathing cycle of the patient.

17. The method according to claim 6, wherein the acoustic spectrum data indicative of the acoustic spectrum of the breathing sounds based on the sound signal is a function of a breathing cycle of the patient.
